(19) United States
(12) Patent Application Publication
(10) Pub. No.: US 2005/0064006 A1
(43) Pub. Date: Mar. 24, 2005

(54) Stents

(75) Inventors: Elisabeth Perzborn, Wuppertal (DE); Jochen Kalbe, Leichlingen (DE); Wolfram Ledwoch, Langenfeld (DE); Didier Meulien, Wuppertal (DE)

Correspondence Address: Jeffrey M Greenman;Bayer Corporation, 400 Morgan Lane, West Haven, CT, 06516, US

(73) Assignee: Bayer Aktiengesellschaft, Leverkusen (DE)

(21) Appl. No.: 10/493,552

(22) PCT Filed: Oct. 11, 2002
(86) PCT No.: PCT/EP02/11402

(30) Foreign Application Priority Data
Oct. 24, 2001 (DE) 101 52 460.9

(51) Int. Cl. A61F 2/06

(52) U.S. Cl. 424/423; 623/1.42; 514/822

(57) ABSTRACT

The invention concerns stents containing compounds of formula (I) and methods for making said stents as well as their use.

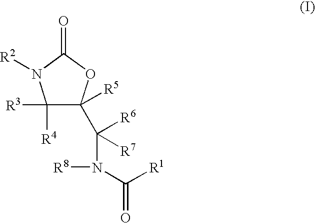

The present invention relates to stents comprising coagulation factor Xa, processes for producing these stents and their use, especially for the treatment and/or prophylaxis of thromboses and/or restenoses.

Coronary diseases caused by arteriosclerosis are treated inter alia by the currently usual method of percutaneous transluminal coronary angioplasty (PTCA). For this purpose, a balloon catheter is introduced into the narrowed or blocked artery, which is then widened through expansion of the balloon, and the blood flow is thus restored. A problem in this connection, occurring in about 30% of cases, is the acute reocclusion, occurring immediately after the PTCA (acute restenosis), or the later, subacute (restenosis) reocclusion, of the blood vessel.

The risk of acute restenosis can be reduced by administration of platelet aggregation inhibitors. An additional possibility is mechanical support of the coronary wall by a normally cylindrical and expandable mesh (stent) which is introduced into the diseased vessel and unfolds at the site of the stenosis in order to open the narrowed place and keep it open by supporting the blood vessel wall. Although it is possible by this method to reduce the risk of restenosis slightly, at present there is still no convincing therapy available for subacute restenosis.

Currently employed systemically in stent treatment are anticoagulants such as, for example heparin; platelet aggregation inhibitors such as, for example aspirin, clopidogrel (Plavix) or ticlopidine (Ticlid); or glycoprotein IIb/IIIa antagonists such as, for example, abciximab.

A newer possibility for the treatment of restenosis is local administration of the active ingredient by means of a stent which releases the active ingredient. The combination of active ingredient and stent makes medical treatment and mechanical stabilization possible in one application.

Thus, the combination of stents with anticoagulants makes it possible for the local concentration of active ingredient to be high without unwanted systemic side effects (e.g. hemorrhages or stroke) occurring.

It is possible for this purpose to coat stents with active ingredient-containing coating materials. The active ingredient release takes place by diffusion from the coating or through breakdown of the coating when biodegradable coating systems are used.

Another possibility which has already been described is the preparation of small cavities or micropores in the stent surface, into which the active ingredient or else active ingredient-containing polymeric coating systems are embedded (see, for example, EP-A 0 950 386). An active ingredient-free coating can subsequently be applied. Release takes place by diffusion or degradation or by a combination of the two processes.

In addition, active ingredient-containing stents can be produced by melt embedding the active ingredient in a polymeric carrier, e.g. with the aid of injection molding processes. Release of the active ingredient from these stents usually takes place through diffusion.

Active ingredients particularly suitable for the treatment and/or prophylaxis of thromboses and restenoses after PTCA are coagulation factor Xa inhibitors.

Thus, coagulation factor Xa is involved in the proliferation of vascular smooth muscle cells (VSMC). The migration and proliferation of VSMC following an injury to the endothelium, and the formation of a neointima resulting therefrom, make a major contribution to the development of restenosis and atherosclerosis. Platelets, thrombin and other components of the thrombotic process are important factors in neointima formation. The serine protease thrombin, whose production is modulated by coagulation factor Xa, exerts further cellular effects, in addition to its effect in the plasma coagulation system, via its specific receptor. By this mechanism it activates platelets and acts as strong mitogen for endothelial cells, VSMC, connective tissue cells and macrophages.

The mitogenic effect of coagulation factor Xa takes place indirectly via the platelet-derived growth factor (PDGF) receptor tyrosine kinase pathway and leads to activation of the mitogen-activated protein kinases (MAPK), which are intracellular mediators of cellular proliferation. The VSMC proliferation modulated by coagulation factor Xa influences the reocclusion of vessels and the restenosis following angioplasty.

Thus, it is possible by specific inhibition of coagulation factor Xa to reduce the intimal hyperplasia after vascular endothelial damage, and thus the restenosis rate after successful angioplasty, since the mitogenic effects of coagulation factor Xa so far reduced and/or the production of the potential mitogen thrombin is reduced (M. M. Samama, J. M. Walenga, B. Kaiser, J. Fareed, Specific Factor Xa Inhibitors, in: M. Verstraete, V. Fuster, E. J. Topol (Ed.), Cardiovascular Thrombosis: Thrombocardiology and Thromboneurology, Philadelphia 1998, pp. 175-176).

It has now been found, surprisingly, that oxazolidinones of the formula (I) which act, in particular, as anticoagulants and as selective inhibitors of coagulation factor Xa, and are described in detail in WO 01/47919, are suitable for this type of treatment. The compounds mentioned generally therein and, in particular, those mentioned specifically therein form an express part of the description of the present invention.

The present invention thus relates to stents comprising one or more compounds of the formula (I) 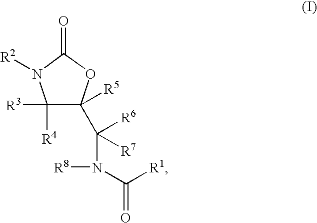
in which:
R¹ is optionally benzo-fused thiophene (thienyl) which may optionally be substituted one or more times;
R² is any organic radical;
R³, R⁴, R⁵, R⁶, R⁷ and R⁸ are identical or different and are hydrogen or (C₁-C₆)-alkyl,
   and the pharmaceutically acceptable salts and/or hydrates thereof.

Preference is given in this connection to stents comprising compounds of the formula (I)

in which

R¹ is optionally benzo-fused thiophene (thienyl) which may optionally be substituted one or more times by a radical from the group of halogen; cyano; nitro; amino; aminomethyl; (C₁-C₈)-alkyl which may in turn be optionally substituted one or more times by halogen; (C₃-C₇)-cycloalkyl; (C₁-C₈)-alkoxy; imidazolinyl; —C(═NH)NH₂; carbamoyl; and mono- and di-(C₁-C₄)-alkylaminocarbonyl,
R² is one of the following groups:
   A-,
   A-M-,
   D-M-A-,
   B-M-A-,
   B-,
   B-M-,
   B-M-B-,
   D-M-B-,
   where:
   the radical “A” is (C₆-C₁₄)-aryl, preferably (C₆-C₁₀)-aryl, in particular phenyl or naphthyl, very particularly preferably phenyl;
   the radical “B” is a 5- or 6-membered aromatic heterocycle which comprises up to 3 heteroatoms and/or hetero chain members, in particular up to 2 heteroatoms and/or hetero chain members, from the series S, N, NO (N-oxide) and O;
   the radical “D” is a saturated or partially unsaturated, mono- or bicyclic, optionally benzo-fused 4- to 9-membered heterocycle which comprises up to three heteroatoms and/or hetero chain members from the series S, SO, SO₂, N, NO(N-oxide) and O;
   the radical “M” is —NH—, —CH₂—, —CH₂CH₂—, —O—, —NH—CH₂—, —CH₂—NH—, —OCH₂—, —CH₂O—, —CONH—, —NHCO—, —COO—, —OOC—, —S—, —SO₂— or a covalent bond;
   where
   the groups “A”, “B” and “D” defined above may in each case optionally be substituted one or more times by a radical from the group of halogen; trifluoromethyl; oxo; cyano; nitro; carbamoyl; pyridyl; (C₁-C₆)-alkanoyl; (C₃-C₇)-cycloalkanoyl; (C₆-C₁₄)-arylcarbonyl; (C₅-C₁₀)-heteroarylcarbonyl; (C₁-C₆)-alkanoyloxymethyloxy; (C₁-C₄)-hydroxyalkylcarbonyl; —COOR²⁷;
   —SO₂R²⁷; —C(NR²⁷R²⁸)═NR²⁹; —CONR²⁸R²⁹; —SO₂NR²⁸R²⁹; —OR³⁰; —NR³⁰R³¹, (C₁-C₆)-alkyl and (C₃-C₇)-cycloalkyl,
   where (C₁-C₆)-alkyl and (C₃-C₇)-cycloalkyl in turn may optionally be substituted by a radical from the group of cyano; —OR²⁷; —NR²⁸R²⁹; —CO(NH)ᵥ(NR²⁷R²⁸) and —C(NR²⁷R²⁸)═NR²⁹,
   where:
   v is either 0 or 1 and
   R²⁷, R²⁸ and R²⁹ are identical or different and are, independently of one another, hydrogen, (C₁-C₄)-alkyl, (C₃-C₇)-cycloalkyl, (C₁-C₄)-alkanoyl, carbamoyl, trifluoromethyl, phenyl or pyridyl,
   and/or
   R²⁷ and R²⁸, or R²⁷ and R²⁹, form, together with the nitrogen atom to which they are bonded, a saturated or partially unsaturated 5- to 7-membered heterocycle having up to three, preferably up to two, identical or different heteroatoms from the group of N, O and S, and
   R³⁰ and R³¹ are identical or different and are, independently of one another, hydrogen, (C₁-C₄)-alkyl, (C₃-C₇)-cycloalkyl, (C₁-C₄)-alkylsulfonyl, (C₁-C₄)-hydroxyalkyl, (C₁-C₄)-aminoalkyl, di-(C₁-C₄)-alkylamino-(C₁-C₄)-alkyl, —CH₂C(NR²⁷R²⁸)═NR²⁹ or —COR³³,
      where
      R³³ is (C₁-C₆)-alkoxy, (C₁-C₄)-alkoxy-(C₁-C₄)-alkyl, (C₁-C₄)-alkoxycarbonyl-(C₁-C₄)-alkyl, (C₁-C₄)-aminoalkyl, (C₁-C₄)-alkoxycarbonyl, (C₁-C₄)-alkanoyl-(C₁-C₄)-alkyl, (C₃-C₇)-cyclo-alkyl, (C₂-C₆)-alkenyl, (C₁-C₈)-alkyl which may optionally be substituted by phenyl or acetyl, or is (C₆-C₁₄)-aryl, (C₅-C₁₀)-heteroaryl, trifluoromethyl, tetrahydrofuranyl or butyrolactone,
R³, R⁴, R⁵, R⁶, R⁷ and R⁸ are identical or different and are hydrogen or (C₁-C⁶)-alkyl,
   and the pharmaceutically acceptable salts and/or hydrates thereof.

Preference is likewise given in this connection to stents comprising compounds of the formula (I)

in which

R¹ is thiophene (thienyl), in particular 2-thiophene, which may optionally be substituted one or more times by halogen, preferably chlorine or bromine, amino, aminomethyl or (C₁-C₈)-alkyl, preferably methyl, where the (C₁-C₈)-alkyl radical may optionally in turn be substituted one or more times by halogen, preferably fluorine,
R² is one of the following groups:
   A-,
   A-M-,
   D-M-A-,
   B-M-A-,
   B-,
   B-M-,
   B-M-B-,
   D-M-B-,
   where:
   the radical “A” is (C₆-C₁₄)-aryl, preferably (C₆-C₁₀)-aryl, in particular phenyl or naphthyl, very particularly preferably phenyl;
   the radical “B” is a 5- or 6-membered aromatic heterocycle which comprises up to 3 heteroatoms and/or hetero chain members, in particular up to 2 heteroatoms and/or hetero chain members, from the series S, N, NO (N-oxide) and O;
   the radical “D” is a saturated or partially unsaturated 4- to 7-membered heterocycle which comprises up to three heteroatoms and/or hetero chain members from the series S, SO, SO₂, N, NO(N-oxide) and O; the radical “M” is —NH—, —CH₂—, —CH₂CH₂—, —O—, —NH—CH₂—, —CH₂—NH—, —OCH₂—, —CH₂O—, —CONH—, —NHCO—, —COO—, —OOC—, —S— or a covalent bond;
   where
   the groups “A”, “B” and “D” defined above may in each case optionally be substituted one or more times by a radical from the group of halogen; trifluoromethyl; oxo; cyano; nitro; carbamoyl; pyridyl; (C₁-C₆)-alkanoyl; (C₃-C₇)-cycloalkanoyl; (C₆-C₁₄)-arylcarbonyl; (C₅-C₁₀)-heteroarylcarbonyl; (C₁-C₆)-alkanoyloxymethyloxy; —COOR²⁷; —SO₂R²⁷; —C(NR²⁷R²⁸)═NR²⁹; —CONR²⁸R²⁹; —SO NR²⁸R²⁹; —OR³⁰; —NR³⁰OR³¹, (C₁-C₆)-alkyl and (C₃-C₇)-cycloalkyl,
   where (C₁-C₆)-alkyl and (C₃-C₇)-cycloalkyl may in turn optionally be substituted by a radical from the group of cyano; —OR²⁷; —NR²⁸R²⁹; —CO(NH)ᵥ(N²⁷R²⁸) and —C(NR²⁷R²⁸)═NR²⁹,
   where:
   v is either 0 or 1, and
   R²⁷, R²⁸ and R²⁹ are identical or different and are, independently of one another, hydrogen, (C₁-C₄)-alkyl or (C₃-C₇)-cycloalkyl,
   and/or
   R²⁷ and R²⁸, or R²⁷ and R²⁹, form, together with the nitrogen atom to which they are bonded, a saturated or partially unsaturated 5- to 7-membered heterocycle having up to three, preferably up to two, identical or different heteroatoms from the group of N, O and S, and
   R³⁰ and R³¹ are identical or different and are, independently of one another, hydrogen, (C₁-C₄)-alkyl, (C₃-C₇)-cycloalkyl, (C₁-C₄)-alkylsulfonyl, (C₁-C₄)-hydroxyalkyl, (C₁-C₄)-aminoalkyl, di-(C₁-C₄)-alkylamino-(C₁-C₄)-alkyl, (C₁-C₄)-alkanoyl, (C₆-C₁₄)-arylcarbonyl, (C₅-C₁₀)-heteroarylcarbonyl, (C₁-C₄)-alkylaminocarbonyl or —CH₂C(NR²⁷R²⁸)═NR²⁹,
   R³, R⁴, R⁵, R⁶, R⁷ and R⁸ are identical or different and are hydrogen or (C₁-C₆)-alkyl,
      and the pharmaceutically acceptable salts and/or hydrates thereof.

Particular preference is given in this connection to stents comprising compounds of the formula (I)

in which

R¹ is thiophene (thienyl), in particular 2-thiophene, which may optionally be substituted one or more times by halogen, preferably chlorine or bromine, or (C₁-C₈)-alkyl, preferably methyl, where the (C₁-C₈)-alkyl radical may in turn optionally be substituted one or more times by halogen, preferably fluorine,
R² is one of the following groups:
   A-,
   A-M-,
   D-M-A-,
   B-M-A-,
   B-,
   B-M-B-,
   D-M-B-,
   where:
   the radical “A” is phenyl or naphthyl, in particular phenyl;
   the radical “B” is a 5- or 6-membered aromatic heterocycle which comprises up to 2 heteroatoms from the series S, N, NO(N-oxide) and O;
   the radical “D” is a saturated or partially unsaturated 5- or 6-membered heterocycle which comprises up to two heteroatoms and/or hetero chain members from the series S, SO, SO₂, N, NO(N-oxide) and O;
   the radical “M” is —NH—, —O—, —NH—CH₂—, —CH₂—NH—, —OCH₂—, —CH₂O—, —CONH—, —NHCO— or a covalent bond;
   where
   the groups “A”, “B” and “D” defined above may in each case optionally be substituted one or more times by a radical from the group of halogen; trifluoromethyl; oxo; cyano; pyridyl; (C₁-C₃)-alkanoyl; (C₆-C₁₀)-arylcarbonyl; (C₅-C₆)-heteroarylcarbonyl; (C₁-C₃)-alkanoyloxymethyloxy; —C(NR²⁷R²⁸)═NR²⁹; —CONR²⁸R²⁹; —SO₂NR²⁸R²⁹; —OH; —NR³⁰OR³¹; (C₁-C₄)-alkyl; and cyclopropyl, cyclopentyl or cyclohexyl,
   where (C₁-C₄)-alkyl and cyclopropyl, cyclopentyl or cyclohexyl may in turn optionally be substituted by a radical from the group of cyano; —OH; —OCH₃; —N²⁸R²⁹; —CO(NH)ᵥ(N²⁷R²⁸) and —C(NR²⁷R²⁸)═NR²⁹,
   where:
   v is either 0 or 1, preferably 0, and
   R²⁷, R²⁸ and R²⁹ are identical or different and are, independently of one another, hydrogen, (C₁-C₄)-alkyl or else cyclopropyl, cyclopentyl or cyclohexyl,
      and/or
   R²⁷ and R²⁸, or R²⁷ and R²⁹, may form, together with the nitrogen atom to which they are bonded, a saturated or partially unsaturated 5- to 7-membered heterocycle having up to two identical or different heteroatoms from the group of N, O and S, and
   R³⁰ and R³¹ are identical or different and are, independently of one another, hydrogen, (C₁-C₄)-alkyl, cyclopropyl, cyclopentyl, cyclohexyl, (C₁-C₄)-alkylsulfonyl, (C₁-C₄)-hydroxyalkyl, (C₁-C₄)-aminoalkyl, di-(C₁-C₄)-alkylamino-(C₁-C₄)-alkyl, (C₁-C₃)-alkanoyl or phenylcarbonyl,
R³, R⁴, R⁵, R⁶, R⁷ and R⁸ are identical or different and are hydrogen or (C₁-C₆)-alkyl,
   and the pharmaceutically acceptable salts and/or hydrates thereof.

Special preference is given in this connection to stents comprising compounds of the formula (I)

in which

R¹ is 2-thiophene which may optionally be substituted in position 5 by a radical from the group chlorine, bromine, methyl or trifluoromethyl,
R² is one of the following groups:
   A-,
   A-M-,
   D-M-A-,
   B-M-A-,
   B-,
   B-M-,
   B-M-B-,
   D-M-B-,
   where:
   the radical “A” is phenyl or naphthyl, in particular phenyl;
   the radical “B” is a 5- or 6-membered aromatic heterocycle which comprises up to 2 heteroatoms from the series S, N, NO(N-oxide) and O;
   the radical “D” is a saturated or partially unsaturated 5- or 6-membered heterocycle which comprises a nitrogen atom and optionally a further heteroatom and/or hetero chain member from the series S, SO, SO₂ and 0; or up to two heteroatoms and/or hetero chain members from the series S, SO, SO₂ and O;
   the radical “M” is —NH—, —O—, —NH—CH₂—, —CH₂—NH—, —OCH₂—, —CH₂O—, —CONH—, —NHCO— or a covalent bond;
   where
   the groups “A”, “B” and “D” defined above may in each case optionally be substituted one or more times by a radical from the group of halogen; trifluoromethyl; oxo; cyano; pyridyl; (C₁-C₃)-alkanoyl; (C₆-C₁₀)-arylcarbonyl; (C₅-C₆)-heteroarylcarbonyl; (C₁-C₃)-alkanoyloxymethyloxy; —CONR²⁸R²⁹; —SO₂NR²⁸R²⁹; —OH; —NR³⁰OR³¹; (C₁-C₄)-alkyl; and cyclopropyl, cyclopentyl or cyclohexyl,
   where (C₁-C₄)-alkyl and cyclopropyl, cyclopentyl or cyclohexyl may in turn optionally be substituted by a radical from the group of cyano; —OH; —OCH₃; —NR²⁸R²⁹; —CO(NH)ᵥ(NR²⁷R²⁸) and —C(NR²⁷R²⁸)═NR²⁹,
   where:
   v is either 0 or 1, preferably 0, and
   R²⁷, R²⁸ and R²⁹ are identical or different and are, independently of one another, hydrogen, (C₁-C₄)-alkyl or else cyclopropyl, cyclopentyl or cyclohexyl,
      and/or
   R²⁷ and R²⁸, or R²⁷ and R²⁹, may form, together with the nitrogen atom to which they are bonded, a saturated or partially unsaturated 5- to 7-membered heterocycle having up to two identical or different heteroatoms from the group of N, O and S, and
   R³⁰ and R³¹ are identical or different and are, independently of one another, hydrogen, (C₁-C₄)-alkyl, cyclopropyl, cyclopentyl, cyclohexyl, (C₁-C₄)-alkylsulfonyl, (C₁-C₄)-hydroxyalkyl, (C₁-C₄)-aminoalkyl, di-(C₁-C₄)-alkylamino-(C₁-C₄)-alkyl, (C₁-C₃)-alkanoyl or phenylcarbonyl,
R³, R⁴, R⁵, R⁶, R⁷ and R⁸ are identical or different and are hydrogen or (C₁-C₄)-alkyl,
   and the pharmaceutically acceptable salts and/or hydrates thereof.

Very particular preference is given in this connection to stents comprising compounds of the formula (I)

in which

R¹ is 2-thiophene which is substituted in position 5 by a radical from the group of chlorine, bromine, methyl or trifluoromethyl,
R² is D-A-:
   where:
   the radical “A” is phenylene;
   the radical “D” is a saturated 5- or 6-membered heterocycle which is linked via a nitrogen atom to “A”,
   which has a carbonyl group in direct vicinity to the linking nitrogen atom, and in which a ring carbon member may be replaced by a heteroatom from the series S, N and O;
   where
   the group “A” defined above may optionally be substituted once or twice in the meta position relative to the linkage to the oxazolidinone by a radical from the group of fluorine, chlorine, nitro, amino, trifluoromethyl, methyl or cyano,
R³, R⁴, R⁵, R⁶, R⁷ and R⁸ are hydrogen,
   and the pharmaceutically acceptable salts and/or hydrates thereof.

Very particular preference is likewise given in this connection to a stent comprising the compound of example 44 of WO 01/47919 having the following formula 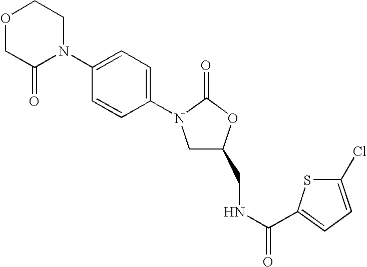
and the pharmaceutically acceptable salts and/or hydrates thereof.

Concerning the disclosure of compounds of the formula (I), for example relating to their preparation, express reference is made to the disclosure in WO 01/47919.

The present invention describes the use of one or more compounds of the formula (I), where appropriate in combination with one or more other active ingredients, for producing a release system comprising medicinal substance(s), in particular a stent comprising medicinal substance(s).

In addition, the present invention describes a release system, in particular a stent, which comprises one or more compounds of the formula (I), where appropriate in combination with one or more other active ingredients, and which makes targeted release of one or more compounds of the formula (I), and of other active ingredients present where appropriate, at the site of action (drug targeting) possible, and are thus suitable for the prophylaxis and/or treatment of restenosis and/or thromboses, in particular after PTCA.

The present invention likewise describes a method for the treatment and/or prophylaxis of thromboses and/or restenosis using one or more compounds of the formula (I) in combination with a stent. In this use it is possible for the compounds of the formula (I) to be employed either systemically or, preferably, in the form of a stent comprising compounds of the formula (I).

Whereas it is not possible with the active ingredients and stents currently available to achieve an adequate success of therapy in all cases, the novel combination of compounds of formula (I) with a stent makes more effective treatment and/or prophylaxis of thromboses and/or restenosis possible. Local administration of compounds of the formula (I) in combination with a stent makes it possible to reduce the dose of the medicinal substance necessary to prevent thromboses and/or restenosis. It is thus possible to minimize undeplored systemic effects. At the same time, the local concentration can be increased and thus the efficacy enhanced.

It is moreover possible, in addition to the administration according to the invention, for a systemic and/or local administration of other active ingredients suitable for the treatment and/or prophylaxis of thromboses and/or restenosis to take place, such as, for example and preferably, abciximab, eptifibatide, tirofiban, acetylsalicylic acid, ticlopidine or clopidogrel. Additional systemic treatment with compounds of the formula (I) is preferred, especially by oral administration.

Release systems comprising the compounds of the invention of the formula (I) are produced by using conventional stents where the basic body of the stent consists either of metals or undegradable plastics such as, for example and preferably, polyethylene, polypropylene, polycarbonate, polyurethane and/or polytetrafluoro-ethylene (PTFE). In addition, stents with various designs of the metal mesh, which make various surfaces and folding principles possible and as described, for example, in WO 01/037761, WO 01/037892 are used as basic body of the stent.

These stents are coated and/or filled with compounds of the formula (I). An alternative possibility in the case of nonmetallic stents is to incorporate compounds of the formula (I) directly into the material used to produce the stents.

Carrier materials are mixed with the compounds of the formula (I) for the coating or filling. Carrier materials used for this purpose are preferably polymeric carriers, in particular biocompatible, nonbiodegradable polymers or polymer mixtures, such as, for example and preferably, polyacrylates and copolymers thereof such as, for example and preferably, poly(hydroxyethyl)methylmethacrylates; polyvinyl-pyrrolidones; cellulose esters and ethers; fluorinated polymers such as, for example and preferably, PTFE; polyvinyl acetates and copolymers thereof; crosslinked and uncrosslinked polyurethanes, polyethers or polyesters; polycarbonates; polydimethyl-siloxanes. As an alternative, biocompatible, biodegradable polymers or polymer mixtures such as, for example and preferably, polymers or copolymers of lactide and glycolide, or of caprolactone and glycolide; other polyesters, polyorthoesters; polyanhydrides; polyamino acids; polysaccharides; polyiminocarbonates; polyphosphazenes and poly(ether-ester) copolymers are also used as polymeric carriers.

Also suitable as polymeric carriers are mixtures of biodegradable and/or non-biodegradable polymers. The rate of release of the active ingredient is adjusted optimally through these mixtures.

Coated or filled stents are produced by dissolving the mixtures of compounds of the formula (I) and carrier, preferably in suitable solvents. These solutions are then applied to the stent by various techniques such as, for example, spraying, dipping or brush-coating. Subsequent or simultaneous removal of the solvent results in the stent provided with the active ingredient-containing coating. An alternative possibility is also for mixtures of compounds of the formula (I) and carrier to be melted and applied by the same application methods.

The stents are preferably pretreated in order to enlarge the outer and/or inner surface area of the stent. This increases the loading potential and larger amounts of coating (active ingredient/polymer) can be applied. Various etching techniques, but also treatments with ionizing radiation, for example, are used for pretreatment of the stents. It is likewise possible to produce micropores or cavities in the stents with the aid of various techniques.

The active ingredient contents of the stents coated or filled with compounds of the formula (I) are usually from 0.001% by weight to 50% by weight, preferably from 0.01% by weight to 30% by weight, particularly preferably 0.1% by weight to 15% by weight.

In the case of nonmetallic stents, the compounds of the formula (I) can also be incorporated directly for example as melt embedding in the basic body of the stent. In theses cases, active ingredient-containing polymeric carrier materials are processed by conventional methods, for example by injection molding processes, to give the final active ingredient-containing form. In these cases, the active ingredient is usually released by diffusion.

The active ingredient contents of stents with embedded compounds of the formula (I) are usually from 0.001% by weight to 70% by weight, preferably from 0.01% by weight to 50% by weight, particularly preferably 0.1% by weight to 30% by weight.

The stents comprising compounds of the formula (I) are, where appropriate, additionally coated with a membrane. This membrane serves, for example and preferably, for controlling the release of medicinal substances and/or for protecting the active ingredient-containing stents from external influences.

1. A stent comprising one or more compounds of the formula (I) 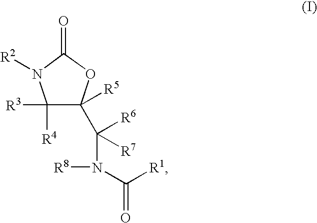
in which
R¹ is 2-thiophene which is substituted in position 5 by a radical from the group of chlorine, bromine, methyl, and trifluoromethyl,
R² is D-A-:
where:
the radical “A” is phenylene;
the radical “D” is a saturated 5- or 6-membered heterocycle which
is linked via a nitrogen atom to “A”,
which has a carbonyl group directly adjacent to the linking nitrogen atom, and
in which a ring carbon member may be replaced by a heteroatom from the series S, N and O;
where
the group “A” defined above may optionally be substituted once or twice in the meta position relative to the linkage to the oxazolidinone by a radical from the group of fluorine, chlorine, nitro, amino, trifluoromethyl, methyl and cyano, and
R³, R⁴, R⁵, R⁶, R⁷ and R⁸ are hydrogen,
or a pharmaceutically acceptable salt, hydrate, salt of a hydrate or hydrate of a salt thereof.

2. The stent of claim 1, characterized in that the compound is 5-chloro-N-({(5S)-2-oxo-3-[4-(3-oxo-4-morpholinyl)phenyl]-1,3-oxazolidin-5-yl}-methyl)-2-thiophenecarboxamide of the formula 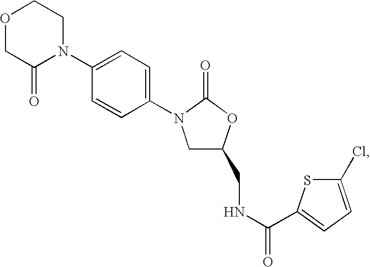
or a pharmaceutically acceptable salt, hydrate, salt of a hydrate or hydrate of a salt thereof.

3. The stent of claim 1, wherein the stent is coated with an additional membrane.

4. The stent of claim 1, comprising at least one other active ingredient.

5. (canceled)

6. (canceled)

7. (canceled)

8. A method of treating or preventing restenosis or thromboses, comprising administering to a patient in need thereof a stent comprising an effective amount of a compound of formula (I) as defined in claim 1.

9. A process for producing a stent, comprising coating or filling a stent with one or more compounds of the formula (I) as defined in claim 1.

10. A process for producing a stent, comprising shaping into a stent, polymeric carrier materials comprising one or more compounds of the formula (I) as defined in claim 1.

11. A method for treating a patient with restenoic arteries, comprising simultaneously administering one or more compounds of the formula (I) as defined in claim 1, and the stent of claim 1.

12. The method as claimed in claim 11, characterized in that compounds of the formula (I) as defined in claim 1 are present in or on the stent and are released locally.

13. A method for treating or preventing restenosis or thromboses, comprising administering to a patient in need thereof the stent of claim 1 in combination with local or systemic administration of other active ingredients suitable for the treatment or prophylaxis of restenosis or thrombosis.

14. A method for treating or preventing restenosis or thromboses, comprising administering to a patient in need thereof the stent of claim 1 in combination with systemic administration of compounds of the formula (I) as defined in claim 1.

15. The method of claim 8, wherein the thromboses are thromboses after PTCA.

16. The method of claim 8, wherein the restenosis is restenosis after PTCA.
